(19) United States
(12) Patent Application Publication
(10) Pub. No.: US 2016/0138061 A1
(43) Pub. Date: May 19, 2016

(54) FATTY ACID AND DERIVATIVES PRODUCTION

(71) Applicants: Thomas HAAS, Muenster (DE); Markus Poetter, Shanghai (CN); Steffen Schaffer, Herten (DE)

(72) Inventors: Thomas HAAS, Muenster (DE); Markus Poetter, Shanghai (CN); Steffen Schaffer, Herten (DE)

(73) Assignee: Evonik Degussa GmbH, Essen (DE)

(21) Appl. No.: 14/943,333

(22) Filed: Nov. 17, 2015

(30) Foreign Application Priority Data
Nov. 17, 2014 (EP) 14193474

(51) Int. Cl. C12P 7/64

(52) U.S. Cl. CPC C12P 7/6409

(57) ABSTRACT

At least one fatty acid and/or derivative thereof is produced from a gas containing H₂, CO₂, and O₂ by providing a genetically modified hydrogen oxidizing bacterium in an aqueous medium; and contacting the aqueous medium with the gas containing H₂, CO₂ and O₂ in a weight ratio of 20 to 70 (H₂): 10 to 45 (CO₂): 5 to 35 (O₂); wherein the fatty acid contains at least 5 carbon atoms and wherein the hydrogen oxidizing bacterium is genetically modified relative to the wild type bacterium to increase the expression of enzyme E₁ that is capable of catalyzing the conversion of acetyl CoA to acyl ACP via malonyl coA and to increase the expression of enzyme E₂ that is capable of catalyzing the conversion of Acyl ACP to the fatty acid.

### BACKGROUND OF THE INVENTION

1. Field of the Invention

The present invention relates to fatty acid production through biotechnology. In particular, the present invention relates to the biotechnological production of a fatty acid with more than 5 carbon atoms from a renewable source.

2. Discussion of the Background

Fatty acids and/or triacylglycerides have various uses in a myriad of industries. In the food industry for example, fatty acids and/or triacylglycerides can be used in animal feed and to supplement nutrition. Fatty acids and/or triacylglycerides can also be used in the cosmetic and pharmacological field. These applications may either require free fatty acids or triacylglycerides. The natural source of these fatty acids and/or triacylglycerides is rather limited.

For example, various polyunsaturated fatty acids (PUFA) and PUFA-containing triglycerides are mainly obtained from microorganisms such as Mortierella and Schizochytrium or from oil-producing plants such as soybean or oilseed rape, algae such as Crypthecodinium or Phaeodactylum and others, where they are usually obtained in the form of their triacylglycerides. The free PUFA are usually prepared from the triacylglycerides by hydrolysis. However, long chain polyunsaturated fatty acids cannot be efficiently isolated from natural oil crop plants.

Traditionally, fatty acids and/or triacylglycerides produced at an industrial scale start with materials derived from petrochemicals. This usually starts with cracking gasoline or petroleum which is bad for the environment. Also, since the costs for these starting materials will be linked to the price of petroleum, with the expected increase in petroleum prices in the future, prices of fatty acids and/or triacylglycerides may also increase relative to the increase in the petroleum prices.

Accordingly, it is desirable to find more sustainable raw materials, other than purely petroleum based, as starting materials for fatty acids and/or triacylglycerides production which also cause less damage to the environment.

A range of biotechnological routes towards production of fatty acids has been described in the past. However, none of them is adequate for the commercial large-scale production of fatty acids from a renewable energy source owing to low yields, insufficient purities and the need for multi-step purification procedures. In particular, only a small proportion of the carbon substrates fed to biotechnologically useful organisms is actually converted to the sought-after product, whilst much of it is consumed by reactions of the primary metabolism.

Another problem associated with biotechnological routes is the fact that a mixture of products is obtained and thus the composition is difficult to control. More specifically, a range of fatty acids may be produced, even though production of a single adduct may be desirable. Since the mixture comprises compounds highly related in terms of chemical structure, purifying or at least enriching a single component in an efficient and straightforward manner is usually beyond technical feasibility.

Accordingly, there is a need in the art for a more efficient means of production of fatty acids from a renewable energy source.

### SUMMARY OF THE INVENTION

The method according to any aspect of the present invention attempts to solve the problems mentioned above.

The present invention therefore relates to a method for producing at least one fatty acid and/or derivative thereof from a gas comprising H₂, CO₂, and O₂, the method comprising:
(a) providing a genetically modified hydrogen oxidizing bacterium in an aqueous medium; and
(b) contacting the aqueous medium with said gas comprising H₂, CO₂, and O₂, in a weight ratio of 20 to 70:10 to 45:5 to 35;

wherein the fatty acid comprises at least 5 carbon atoms; and

wherein the hydrogen oxidizing bacterium is genetically modified relative to the wild type bacterium to increase the expression of enzyme E₁ that catalyzes the conversion of acetyl CoA to acyl ACP via malonyl CoA and to increase the expression of enzyme E₂ that catalyzes the conversion of Acyl ACP to the fatty acid.

### DETAILED DESCRIPTION OF THE INVENTION

Any ranges mentioned below include all values and subvalues between the upper and lower limits of the ranges.

In particular, the method according to any aspect of the present invention may be able to use at least one genetically modified hydrogen oxidizing bacteria to convert a carbon source to at least one fatty acid and/or derivative thereof by way of first converting the carbon source to acyl CoA and then converting the acyl CoA to malonyl coA and then finally converting the malonyl coA to at least one fatty acid with more than 5 carbon atoms. This method according to any aspect of the present invention allows for the production of fatty acid and derivatives thereof in the absence of energy-rich organic compounds. Also, the method according to any aspect of the present invention provides an efficient biotechnological route towards the production of fatty acid and derivatives thereof. In the method according to any aspect of the present invention, more carbon atoms in the fatty acid and derivatives thereof obtained are derived from carbon dioxide molecules, i.e. the proportion of carbon atoms derived from carbon dioxide rather than from any other organic molecules, for example alcohols or carbohydrates, is higher using the method according to any aspect of the present invention compared to other methods known in the art.

According to one aspect of the present invention, there is provided a method of producing at least one fatty acid and/or derivative thereof from a gas comprising H₂, CO₂ and/or O₂ the method comprising the steps of:
(a) providing a genetically modified hydrogen oxidizing bacterium in an aqueous medium; and
(b) contacting the aqueous medium with a gas comprising H₂, CO₂ and/or O₂ in a weight ratio of 20 to 70:10 to 45:5 to 35;

wherein the fatty acid comprises at least 5 carbon atoms and

wherein the hydrogen oxidizing bacterium is genetically modified relative to the wild type bacterium to increase the expression of enzyme E₁ that is capable of catalyzing the conversion of acetyl CoA to acyl ACP via malonyl coA and to increase the expression of enzyme E₂ that is capable of catalyzing the conversion of Acyl ACP to the fatty acid.

The genetically modified hydrogen oxidizing bacterium according to any aspect of the present invention refers to recombinant hydrogen oxidizing that may be engineered to yield various types of fatty acid and derivatives thereof including, but not limited to, short chain alcohols such as ethanol, propanol isopropanol and butanol, fatty alcohols, fatty acid esters, hydrocarbons, wax esters and the like.

In one example, the disclosure provides a method for modifying a hydrogen oxidizing bacterium so that it produces, and optionally releases, fatty acids and/or derivatives thereof generated from a renewable carbon source. Such hydrogen oxidizing bacteria are genetically engineered, for example, by introducing an exogenous DNA sequence encoding one or more proteins capable of metabolizing a renewable carbon source to produce, and in some examples secrete, a fatty acid derivative. The modified hydrogen oxidizing bacteria can then be used in a fermentation process to produce useful fatty acids and/or derivatives thereof using the renewable carbon source (biomass) as a starting material. In some examples, existing genetically tractable hydrogen oxidizing bacteria are used because of the ease of engineering its pathways for controlling growth, production and reducing or eliminating side reactions that reduce biosynthetic pathway efficiencies. Also, such modified hydrogen oxidizing bacteria can be used to consume renewable carbon sources in order to generate fuels that can be directly used as biofuels, without the need for special methods for storage, or transportation. In other examples, microorganisms that naturally produce hydrocarbons are engineered to overproduce hydrocarbons by expressing exogenous nucleic acid sequences that increase fatty acid production.

The method according to any aspect of the present invention may be used to produce fatty acid and/or derivatives thereof having defined carbon chain length, branching, and saturation levels. In particular examples, the production of homogeneous products decreases the overall cost associated with fermentation and separation. In particular, the fatty acid comprises at least 5 carbon atoms. For example, the fatty acid may be saturated or unsaturated. The fatty acid may comprise 5, 6, 7, 8, 9, 10, 11, 12, 13, 14, 15, 16, 17, 18, 19, 20 carbon atoms. In some examples, the fatty acid may comprise more than 20 carbon atoms. More in particular, the fatty acid and/or derivatives thereof may comprise a carbon chain that is at least 8, 10, 12, 14, 16, 18, 20, 22, 24, 26, 28, 30, 32, or 34 carbons long. In some examples at least 50%, 60%, 70%, 80%, 85%, 90%, or 95% of the fatty acid and/or derivative product made contains a carbon chain that is 8, 10, 12, 14, 16, 18, 20, 22, 24, 26, 28, 30, 32, or 34 carbons long. In yet other examples, at least 60%, 70%, 80%, 85%, 90%, or 95% of the fatty acid and/or derivative product contain 1, 2, 3, 4, or 5, points of unsaturation. In particular, the method according to any aspect of the present invention may result in the production of a combination of fatty acids. More in particular, the result may comprise a combination of at least 2, 3, 4, 5 or 6 fatty acids produced.

According to any aspect of the present invention, the carbon source used in step (b) may be any renewable carbon source. In particular, the carbon source may comprise H₂, CO₂ and/or O₂. More in particular, the carbon source comprises oxygen gas allowing for production of fatty acids in aerobic conditions.

The partial pressure of hydrogen in the H₂, CO₂ and O₂ gas introduced in step (b) may be 0.1 to 100 bar, 0.2 to 10 bar, particularly 0.5 to 4 bar. The partial pressure of carbon dioxide in the H₂, CO₂ and O₂ containing gas introduced in step (b) may be 0.03 to 100 bar, particularly 0.05 to 1 bar, more in particular 0.05 to 0.3 bar. The partial pressure of oxygen in the H₂, CO₂ and O₂ gas introduced in step (b) may be 0.001 to 100 bar, particularly 0.04 to 1 bar, more in particular 0.04 to 0.5 bar. In particular, the source of H₂ and/or CO₂ in step (b) may be synthesis gas. The synthesis gas may be used in combination with oxygen to be the source of H₂, CO₂ and O₂ gas introduced in step (b). More in particular, the H₂, CO₂ and O₂ gas introduced in step (b) according to any aspect of the present invention may comprise synthesis gas.

Synthesis gas can be provided from the by-product of coal gasification, for example. Consequently, the hydrogen oxidizing bacterium converts a substance that is a waste product, into a valuable raw material.

Alternatively, synthesis gas may be provided by the gasification of widely available, low-cost agricultural raw materials for the process according to any aspect of the present invention. There are numerous examples of raw materials that can be converted into synthesis gas, as almost all forms of vegetation can be used for this purpose. Examples of raw materials are perennial grasses such as miscanthus, corn residues, processing waste such as sawdust. In general, the synthesis gas may be obtained in a gasification apparatus of dried biomass with primary products such as H₂, CO₂ and O₂, mainly through pyrolysis, partial oxidation and/or steam reforming. Usually a portion of the product gas is processed in order to optimize product yields, and to avoid formation of tar. Cracking of the undesired tar and CO in the synthesis gas may be carried out using lime and/or dolomite. These processes are described in detail in Reed, T B, 1981. Mixtures of different sources for the generation of synthesis gas can also be used.

In particular, the carbon contained in the synthesis gas in step (b) comprises at least 50% by weight, or at least 70%, 90% by weight of the carbon of all carbon sources that is available to the hydrogen oxidizing bacteria in process step (b), wherein the weight percent of coal substance relate to the carbon atoms. Other carbon sources besides CO₂ from the synthesis gas may be available to the hydrogen oxidizing bacteria, for example in the form of carbohydrates in the aqueous medium.

More CO₂ sources such as flue gas, petroleum refinery gases, product of yeast fermentation or clostridial fermentation, exhaust gases from the gasification of cellulose-containing materials or coal gasification and the like may be used as the source of CO₂ in the H₂, CO₂ and O₂ gas introduced in step (b).

In particular, the gas comprising H₂, CO₂ and/or O₂ introduced in step (b) comprises a weight ratio of 20 to 70:10 to 45:5 to 35 respectively. The ratio of H₂ and O₂ may vary depending on the source. For example, the weight ratio of H₂, to O₂ may be 2:1, 4:1, 5:1 and the like.

The term “hydrogen oxidizing bacterium” may be used interchangeably with the term “knallgas bacterium”, and refers to any bacterium capable of oxidizing hydrogen, using oxygen as a terminal electron acceptor, and of fixing carbon dioxide under aerobic conditions. In particular, the knallgas bacterium may be selected from the group consisting of Methanobacterium, Acetobacterium, Desulfovibrio, Desulfomonas, Paracoccus, Achromobacter, Alcaligenes, Pseudomonas, Nocardia and Cupriavidus. More in particular, the knallgas bacterium may be a Cupriavidus strain, even more in particular, Cupriavidus necator H16. Exemplary knallgas bacteria may comprise of, but are not limited to Acidovorax facilis, Acidovorax sp., Alcaligenes eutropha, Alcaligenes sp., Bradyrhizobium japonicum, Bradyrhizobium sp., Cupriavidus necator DSM 531, Heliobacter sp., Hydrogenobacter sp., Hydrogenobacter thermophilus, Hydrogenomonas eutropha, Hydrogenomonas pantotropha, Hydrogenomonas sp., Hydrogenomonas facilis, Hydrogenophaga sp., Hydrogenovibrio marinus (strain MH-110), Hydrogenovibrio sp., Oxyhydrogen microorganism, Pseudomonas hydrogenothermophila, Pseudomonas hydrogenovora, Pseudomonas sp., Ralstonia eutropha, Ralstonia sp., Rhizobium japonicum, Rhizobium sp., Rhodococcus opacus, Rhodococcus sp., Rhodopseudomonas acidophila, Rhodopseudomonas blastica, Rhodopseudomonas capsulata, Rhodopseudomonas palustris, Rhodopseudomonas sheroides, Rhodopseudomonas sulfoviridis, Rhodopseudomonas viridis, Rhodoseudomonas sp., Rhodospirillum rubrum, Rhodospirillum sp., Thiocapsa roseopersicina, Thiocapsus sp., Variovorax paradoxus, Variovorax sp., Xanthobacter sp., Hydrogenothermus marinus, Ralstonia eutropha, Cupriavidus necator, Alcaligenes eutrophus, Alcaligenes paradoxus, Paracoccus denitrificans, Pseudomonas facilis, Pseudomonas flava, Pseudomonas palleronii, Pseudomonas saccharophila, and Rhodococcus sp. (Nocardia opaca), Pseudomonas pseudoflava. In one example, the knallgas bacterium may be a bacterium genetically modified to be a knallgas bacterium, whilst the corresponding wild type strain is not. In this case, the bacterium may be any organism amenable to such modifications, for example E. coli.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that code for a corresponding enzyme with increased activity and optionally by combining these measures. Genetically modified cells or organisms used according to any aspect of the present invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extra-chromosomally replicating vector.

The genetically modified cell may be genetically different from the wild type cell. The genetic difference between the genetically modified cell according to any aspect of the present invention and the wild type cell may be in the presence of a complete gene, amino acid, nucleotide etc. in the genetically modified cell that may be absent in the wild type cell. In one example, the genetically modified cell according to any aspect of the present invention may comprise enzymes that enable the cell to produce at least one fatty acid and/or acyl coenzyme A thereof; and convert the fatty acid and/or acyl coenzyme A thereof to the fatty acid ester. The wild type cell relative to the genetically modified cell of the present invention may have none or no detectable activity of the enzymes that enable the genetically modified cell to produce at least one fatty acid and/or derivative thereof.

The phrase “wild type” as used herein in conjunction with a cell may denote a cell with a genome make-up that is in a form as seen naturally in the wild. The term may be applicable for both the whole cell and for individual genes. The term “wild type” therefore does not include such cells or such genes where the gene sequences have been altered at least partially by man using recombinant methods.

A skilled person would be able to use any method known in the art to genetically modify a cell. According to any aspect of the present invention, the genetically modified cell may be genetically modified so that in a defined time interval, within 2 hours, in particular within 8 hours or 24 hours, it forms at least twice, especially at least 10 times, at least 100 times, at least 1000 times or at least 10000 times more fatty acid and/or thereof than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (fatty acid, acyl coenzyme A thereof and the respective fatty acid ester) in the nutrient medium.

According to any aspect of the present invention, the hydrogen oxidizing bacterium may be genetically modified relative to the wild type bacterium to increase the production of acyl-ACP or acyl-CoA, reduce the catabolism of fatty acid derivatives and intermediates, or to reduce feedback inhibition at specific points in the biosynthetic pathway. In one example, cellular resources can also be diverted to aid in the overproduction of fatty acids, for example the lactate, succinate and/or acetate pathways can be attenuated, and acetyl-CoA carboxylase (ACC) can be overexpressed. In particular, the expression of enzyme E₁ that may be capable of catalyzing the conversion of acetyl CoA to acyl ACP via malonyl CoA may be increased in the hydrogen oxidizing bacterium according to any aspect of the present invention. In one example, the hydrogen oxidizing bacterium according to any aspect of the present invention may also comprise an increase in the expression of enzyme E₂ that may be capable of catalyzing the conversion of Acyl ACP to the fatty acid. The the hydrogen oxidizing bacterium according to any aspect of the present invention may comprise increase in the expression of E₁ and E₂.

The genetically modified hydrogen oxidizing bacterium according to any aspect of the present invention may be cultured in a desired environment, for example one with limited glycerol (less than 1% w/v in the culture medium). As such, these hydrogen oxidizing bacteria will have increased acetyl-CoA production levels. With an increase in acetyl-CoA production levels, malonyl-CoA production may also be increased by genetically modifying the hydrogen oxidizing bacteria as described above, with DNA encoding accABCD (acetyl CoA carboxylase, for example accession number AAC73296, EC 6.4.1.2) included in the plasmid synthesized de novo. Fatty acid overproduction can be achieved by further including DNA encoding lipase (for example accession numbers CAA89087, CAA98876) in the plasmid synthesized de novo.

In some examples, acetyl-CoA carboxylase (ACC) is over-expressed to increase the intracellular concentration thereof by at least 2-fold, such as at least 5-fold, or at least 10-fold, for example relative to native expression levels.

In one example, the plsB (for example accession number AAC77011) D311E mutation can be used to remove limitations on the pool of acyl-CoA.

Overexpression of a sfa gene (suppressor of Fab A, for example with accession number AAN79592) may also be included in the genetically modified hydrogen oxidizing bacterium to increase production of monounsaturated fatty acids (Rock et al, 1996).

In particular, E₁ may be selected from a group of enzymes that are capable of catalyzing the conversion of acetyl CoA to acyl ACP via malonyl CoA. E₁ may thus be selected from the group consisting of E₁ₐ accA (EC 6.4.1.2), E_{1b} accB (EC 6.4.1.2), E_{1c} accC (EC 6.3.4.14), E_{1d} accD (EC 6.4.1.2) and combinations thereof. More in particular, the genetically modified hydrogen oxidizing bacterium according to any aspect of the present invention may be genetically modified to increase the expression of E₁ₐ, E_{1b}, E_{1c}, E₁ₐ, E₁ₐE_{1b}, E₁ₐE_{1c}, E₁ₐE_{1d}, E_{1b}E_{1c}, E_{1b}E_{1d}, E_{1c}E_{1d}, E₁ₐE_{1b}E_{1c}, E₁ₐE_{1b}E_{1d}, E_{1b}E_{1c}E_{1d} or E₁ₐE_{1b}E_{1c}E_{1d}.

In particular, the genetically modified hydrogen oxidizing bacterium according to any aspect of the present invention may be modified to overexpress at least one enzyme E₂ capable of catalyzing the conversion of acyl ACP to fatty acid, possibly free fatty acid. More in particular, E₂ may be a thioesterase. Even more in particular, E₂ may be selected from the group consisting of acyl-ACP thioesterase E₂ₐ, acyl-CoA thioesterase E_{2b}, acyl-thioesterase E_{2c} and the like. In particular, E₂ₐ (EC 3.1.2.14 or EC 3.1.2.22) may be any enzyme that is capable of catalyzing the hydrolysis of acyl-ACP thioester; E_{2b} (EC 3.1.2.2, EC 3.1.2.18, EC 3.1.2.19, EC 3.1.2.20 or EC 3.1.2.22) may be any enzyme that is capable of catalyzing the hydrolysis of acyl-CoA thioester; E_{2c} (EC 3.1.2.2, EC 3.1.2.4, EC 3.1.2.18, EC 3.1.2.19, EC 3.1.2.20 or EC 3.1.2.22) may be any enzyme that is capable of catalyzing the conversion of an acyl-thioester with an alcohol to a carboxylic acid ester. E₂ may be chosen to provide homogeneous products.

More in particular, E₂ may be E₂ₐ. For example, C₁₀ fatty acid and derivatives thereof can be produced by attenuating thioesterase C₁₈ (for example accession numbers AAC73596 and POADA1), which uses C18:1-ACP and expressing thioesterase C₁₀ (for example accession number Q39513), which uses C10-ACP. Thus, resulting in a relatively homogeneous population of fatty acids and/or derivatives thereof that have a carbon chain length of 10. In another example, C₁₄ fatty acid and derivatives can be produced by attenuating endogenous thioesterases that produce non-C₁₄ fatty acids and expressing the thioesterase with accession number Q39473, which uses C14-ACP). In yet another example, C₁₂ fatty acid and derivatives thereof can be produced by expressing thioesterases that use C12-ACP (for example accession number Q41635) and attenuating thioesterases that produce non-C12 fatty acids. Examples of E₂ that may be used to produce specific fatty acids are provided in Table 1 below. Acetyl CoA, malonyl CoA, and fatty acid overproduction can be verified using methods known in the art, for example by using radioactive precursors, HPLC, and GC-MS subsequent to cell lysis.

| | | | Preferential |
|---|---|---|---|
| Accession | | | product |
| Number | Source Organism | Gene | produced |
| AAC73596 | E. coli | tesA without | C18:1 |
| | | leader sequence | |
| Q41635 | Umbellularia | fatB | C12:0 |
| | california | | |
| Q39513; | Cuphea hookeriana | fatB2 | C8:0-C10:0 |
| AAC49269 | Cuphea hookeriana | fatB3 | C14:0-C16:0 |
| Q39473 | Chinnamonum | fatB | C14:0 |
| | Camphorum | | |
| CAA85388 | Arabidopsis thaliana | fatB[M141T]* | C16:1 |
| NP 189147; | Arabidopsis thaliana | fatA | C18:1 |
| NP 193041 | | | |
| CAC39106 | Bradyrhiizobium | fatA | C18:1 |
| | japonicum | | |
| AAC72883 | Cuphea hookeriana | fatA | C18:1 |

Table 1. Examples of thioesterases for production of fatty acid from acyl-ACP

According to any aspect of the present invention, the hydrogen oxidizing bacterium may be genetically modified to express E₂ selected from the group consisting of E₂ₐ, E_{2b}, and E_{2c} in combination with E₁ₐ, E_{1b}, E_{1c}, E_{1d}, E₁ₐE_{1b}, E₁ₐE_{1c}, E₁ₐE_{1d}, E_{1b}E_{1c}, E_{1b}E_{1d}, E_{1c}E_{1d}, E₁ₐE_{1b}E_{1c}, E₁ₐE_{1b}E_{1d}, E_{1b}E_{1c}E_{1d} or E₁ₐE_{1b}E_{1c}E_{1d}. More in particular, the hydrogen oxidizing bacterium may be genetically modified to express E₁ₐE_{1b}E_{1c}E_{1d}E₂ₐ.

In one example, E₁ₐ, has sequence identity of at least 50% to a polypeptide of AAC73296 or EG11647; E_{1b} has sequence identity of at least 50% to a polypeptide of EG10275; E_{1c} has sequence identity of at least 50% to a polypeptide of EG10276; and/or E_{1d} has sequence identity of at least 50% to a polypeptide of EG10217 and combinations thereof. E₁ₐ E_{1b}. E_{1c} and E_{1d} used according to any aspect of the present invention may comprise sequence identity of at least 60, 65, 70, 75, 80, 85, 90, 95, 98 or 100% to a polypeptide of any one of sequences of accA (AAC73296 or EG11647), accB (EG10275), accC (EG10276), accD (EG10217) and combinations thereof. In particular, the hydrogen oxidizing bacterium may be genetically modified to at least comprise a functional fragment of any of the polypeptides for catalyzing the conversion of conversion of acetyl CoA to acyl ACP via malonyl coA.

In one example, E₂ has sequence identity of at least 50% to a polypeptide selected from the group consisting of AAC79596, ACC49269, CAA58388, NP189147, NP193041, AAC72883, AAC72881.1, ABB71579.1, CAC19934.1, AAC49180.1 (encoded by SEQ ID No.10), AAC49783.1, AAC49179.1, CAB60830.1, ABB71581.1, AAC49269.1, CAC19933.1, CAA54060.1, AAC72882.1, Q39513.1, AAC49784.1, AB038558.1, AB038555.1, AB038556.1, AB038554.1, ADB79568.1, ADB79569.1, ACQ57188.1, ACQ57189.1, ABK96561.1, ACQ63293.1, ACQ57190.1, Q9SQI3.1, ABU96744.1, ABC47311.1, XP_002324962.1, AAD01982.1, AAB51525.1, ACV40757.1, XP_002309244.1, CBI28125.3, ABD91726.1, XP_002284850.1, XP_002309243.1, XP_002515564.1, ACR56792.1, ACR56793.1, XP_{—} 002892461.1, ABI18986.1, NP_172327.1, CAA85387.1, CAA85388.1, ADA79524.1, ACR56795.1, ACR56794.1, CAN81819.1, ACF17654.1, AAB71729.1, ABH11710.1, ACQ57187.1, AAX51637.1, AAB88824.1, AAQ08202.1, AAB71731.1, AAX51636.1, CAC80370.1, CAC80371.1, AAG43858.1, ABD83939.1, AAD42220.2, AAG43860.1, AAG43861.1, AAG43857.1, AAL15645.1, AAB71730.1, NP_001068400.1, EAY86877.1, NP_001056776.1, XP_002436457.1, NP_001149963.1, ACN27901.1, EAY99617.1, ABL85052.1, XP_002437226.1, NP_001151366.1, ACF88154.1, NP_001147887.1, XP_002453522.1, BAJ99650.1, EAZ37535.1, EAZ01545.1, AAN17328.1, EAY86884.1, EEE57469.1, Q41635.1, AAM09524.1, Q39473.1, NP_001057985.1, AAC49001.1, XP_001752161.1, XP_001770108.1, XP_001784994.1, XP_002318751.1, NP_001047567.1, XP_002322277.1, XP_002299627.1, XP_002511148.1, CBI15695.3, XP_002299629.1, XP_002280321.1, CAN60643.1, XP_002459731.1, XP_{—} 002975500.1, XP_{—} 002962077.1, XP_001773771.1, NP_001151014.1, XP_002317894.1, XP_002971008.1, XP_001774723.1, XP_002280147.1, XP_002526311.1, XP_002517525.1, XP_001764527.1, ABI20759.1, BAD73184.1, XP_002987091.1, XP_002985480.1, CBI26947.3, ABI20760.1, XP_002303055.1, XP_002885681.1, ADH03021.1, XP_002532744.1, EAY74210.1, EEC84846.1, EEE54649.1, AAG35064.1, AAC49002.1, CAD32683.1, ACF78226.1, BAJ96402.1, XP_002462626.1, NP_001130099.1, XP_002462625.1, ABX82799.3, Q42712.1, NP_193041.1, AAB51524.1, NP_189147.1, ABR18461.1, XP_002863277.1, AAC72883.1, AAA33019.1, CBI40881.3, XP_002262721.1, AAB51523.1, NP_001063601.1, ADB79567.1, AAL77443.1, AAL77445.1, AAQ08223.1, AAL79361.1, CAA52070.1, AAA33020.1, CAA52069.1, XP_001785304.1, CAC39106.1, XP_002992591.1, XP_002968049.1, XP_001770737.1, XP_001752563.1, AAG43859.1, XP_002978911.1, XP_002977790.1, ACB29661.1, XP_002314829.1, XP_002991471.1, EAZ45287.1, XP_002986974.1, EEC73687.1, XP_002312421.1, ACJ84621.1, NP_001150707.1, AAD28187.1, XP_001759159.1, XP_001757193.1, XP_002322077.1, ABE01139.1, XP_002447294.1, AAX54515.1, AAD33870.1. In particular, E₂ may be selected from the group consisting of AAC72881.1, ABB71579.1, CAC19934.1, AAC49180.1, AAC49783.1, AAC49179.1, CAB60830.1, ABB71581.1, AAC49269.1, CAC19933.1, CAA54060.1, AAC72882.1, Q39513.1, AAC49784.1, AAC72883.1, Q41635.1, and AAC49001.1 and combinations thereof. E₂ used according to any aspect of the present invention may comprise sequence identity of at least 60, 65, 70, 75, 80, 85, 90, 95, 98 or 100% to a polypeptide of any one of sequences of provided above and combinations thereof. In particular, the hydrogen oxidizing bacterium may be genetically modified to at least comprise a functional fragment of any of the polypeptides for catalyzing the conversion of conversion of acyl ACP to at least one fatty acid.

In some examples, the fatty acid may undergo a further to step to produce at least one fatty acid derivative selected from the group consisting of butanol, fatty alcohols, fatty acid esters, hydrocarbons, wax esters and the like.

In one example, the genetically modified hydrogen oxidizing bacterium according to any aspect of the present invention may be modified to overexpress at least one wax ester synthase (EC 2.3.1.75) (E₂₁). The genetically modified hydrogen oxidizing bacterium may be modified to express E₁ₐE_{1b}E_{1c}E_{1d}E₂E₂₁. In another example, the genetically modified hydrogen oxidizing bacterium according to any aspect of the present invention may be modified to overexpress at least one alcohol acetyltransferase (2.3.1.84) (E₂₂). In yet other examples, the genetically modified hydrogen oxidizing bacterium may be modified to express E₁ₐE_{1b}E_{1c}E_{1d}E₂E₂₁E₂₂, E₁ₐE_{1b}E_{1c}E_{1d}E₂E₂₂ and the like.

In yet other examples, the genetically modified hydrogen oxidizing bacterium according to any aspect of the present invention may be modified to overexpress at least one acyl-CoA reductase (EC 1.2.1.50) (E₂₃) and at least one alcohol dehydrogenase (EC 1.1.1.1) (E₂₄). In particular, the genetically modified hydrogen oxidizing bacterium may be modified to express E₁ₐE_{1b}E_{1c}E_{1d}E₂E₂₃E₂₄, E₁ₐE_{1b}E_{1c}E_{1d}E₂E₂₃, E₁ₐE_{1b}E_{1c}E_{1d}E₂E₂₄ and the like.

In a further example, the genetically modified hydrogen oxidizing bacterium according to any aspect of the present invention may be modified to overexpress at least one fatty alcohol forming acyl-CoA reductase (1.1.1.*) (E₂₅). In particular, the genetically modified hydrogen oxidizing bacterium may be modified to express E₁ₐE_{1b}E_{1c}E_{1d}E₂E₂₅, and the like.

In one example, the genetically hydrogen oxidizing bacterium according to any aspect of the present invention may be modified to increase the expression relative to the wild type bacterium of enzyme E₂₆ a fatty acid methyl transferase (EC 2.1.1.15). In particular, the genetically modified hydrogen oxidizing bacterium may be modified to express E₁ₐE_{1b}E_{1c}E_{1d}E₂E₂₆ and the like.

The genetically modified hydrogen oxidizing bacterium according to any aspect of the present invention may also be modified to have one or more endogenous genes functionally deleted or attenuated to aid in the production of fatty acids and/or derivatives thereof. For example, ackA (EC 2.7.2.1), ackB (EC 2.7.2.1), adhE (EC 1.1.1.1, 1.2.1.10), fabF (EC 2.3.1.179), fabR (accession NP_418398), fadE (EC 1.3.99.3, 1.3.99.-), GST (EC 6.3.2.3), gpsA (EC 1.1.1.94), ldhA (EC 1.1.1.28), pflB (EC EC 2.3.1.54), plsB (EC 2.3.1.15), poxB (EC 1.2.2.2), pta (EC 2.3.1.8), glutathione synthase (EC 6.3.2.3) and combinations thereof.

The genetically modified hydrogen oxidizing bacterium according to any aspect of the present invention may also be modified to have one or more endogenous genes overexpressed. For example, pdh, panK, aceEF (encoding the EIp dehydrogenase component and the E2p dihydrolipoamide acyltransferase component of the pyruvate and 2-oxoglutarate dehydrogenase complexes, Accession Numbers: NP_414656, NP_414657, EC: 1.2.4.1. 2.3.1.61, 2.3.1.12), accABCD/fabH/fabD/fabG/acpP/fabF (Accession Numbers: Q0KCA7, G0EWI2, F8GYI0, G0EWI3, F8GWN5, GOERC9, CAD85557, CAD85558, NP_842277, NP_841683, NP_415613, EC 6.4.1.2 C, EC: 2.3.1.180, 2.3.1.39, 1.1.1.100, 1.6.5.3, 2.3.1.179), genes encoding fatty-acyl-coA reductases (Accession numbers: AAC45217, EC 1.2.1.-), UdhA or similar genes (encoding pyridine nucleotide transhydrogenase, Accession numbers: CAA46822, EC: 1.6.1.1) and genes encoding fatty-acyl-coA reductases (Accession numbers: AAC45217, EC 1.2.1.-).

Fatty acid synthase (FAS) is a group of peptides that catalyze the initiation and elongation of acyl chains (Marrakchi et al., 2002). The acyl carrier protein (ACP) along with the enzymes in the FAS pathway control the length, degree of saturation and branching of the fatty acids produced. Enzymes that can be included in FAS include but are not limited to AccABCD, FabD, FabH, FabG, FabA, FabZ, FabI, FabK, FabL, FabM, FabB, FabF and the like. The genes may be overexpressed or attenuated depending on the specific fatty acid desired to provide the suitable conditions for production of the fatty acid. The precursors of fatty acid production according to any aspect of the present invention are acetyl-CoA and malonyl-CoA. Hydrogen oxidizing bacterium engineered to overproduce these components can serve as the starting point for subsequent genetic engineering steps to provide fatty acid derivatives such as, fatty acid esters, hydrocarbons, fatty alcohols and the like. Several different modifications can be made, either in combination or individually, to the host hydrogen oxidizing bacterium strain to obtain increased acetyl CoA/malonyl CoA/fatty acid and fatty acid derivative production. For example, to increase acetyl CoA production, a plasmid with pdh, panK, aceEF, (encoding the EIp dehydrogenase component and the E2p dihydrolipoamide acyltransferase component of the pyruvate and 2-oxoglutarate dehydrogenase complexes), fabH/fabD/fabG/acpP/fabF, and in some examples additional DNA encoding fatty-acyl-CoA reductases and aldehyde decarbonylases, all under the control of a constitutive, or otherwise controllable promoter, can be constructed. Exemplary Genbank accession numbers for these genes are: pdh (BAB34380, AAC73227, AAC73226), panK (also known as coaA, AAC76952), (E₃ and E₄) aceEF (AAC73227, AAC73226), (E₁₂) fabH (AAC74175), (E₁₀) fabD (AAC74176), (E₁₁) fabG (AAC74177), (E₅) acpP (AAC74178) and fabF (AAC74179).

Other enzymes such as fadE, gpsA, IdhA, pflb, adhE, pta, poxB, ackA, and/or ackB may be knocked-out, or their expression levels can be reduced, in the engineered hydrogen oxidizing bacterium according to any aspect of the present invention by transformation with conditionally replicative or non-replicative plasmids containing null or deletion mutations of the corresponding genes, or by substituting promoter or enhancer sequences. Exemplary Genbank accession numbers for these genes are: fadE (AAC73325), gspA (AAC76632), IdhA (AAC74462), pflb (AAC73989), adhE (AAC74323), pta (AAC75357), poxB (AAC73958), ackA (AAC75356), and ackB (BAB81430).

Even more in particular, the genetically modified hydrogen oxidizing bacterium according to any aspect of the present invention may be modified to overexpress at least one enzyme selected from the group consisting of E₃ aceE (EC 1.2.4.1, 2.3.1.61, 2.3.1.12), E₄ aceF (EC 1.2.4.1, 2.3.4.16, 2.3.1.12), E₅ acpP (AAC74178), E₆ fadD (EC 2.3.1.86), E₇ cerl (EC 4.1.99.5), E₈ fabA (EC4.2.1.60), E₉ fabB (EC 2.3.1.41), E₁₀ fabD (EC 2.3.1.39), E₁₁ fabG (EC 1.1.1.100), E₁₂ fabH (EC 2.3.1.180), E₁₃fabI (EC 1.3.1.9), E₁₄ fabZ (EC 4.2.1.-), E₁₅ lipase (EC 3.1.1.3), E₁₆ malonyl-CoA decarboxylase (EC 4.1.1.9, 4.1.1.41), E₁₇ panD (EC 4.1.1.11), E₁₈ panK (EC 2.7.1.33), E₁₉ pdh (EC 1.2.4.1), E₂₀ udhA (EC 1.6.1.1) and combinations thereof. In one example, the genetically modified hydrogen oxidizing bacterium according to any aspect of the present invention may be modified to overexpress E₁ₐ, E_{1b}, E_{1c} and/or E_{1d} and combinations thereof as mentioned above in combination with at least one enzyme selected from E₃, E₄, E₅, E₆, E₇, E₈, E₉, E₁₀, E₁₁, E₁₂, E₁₃, E₁₄, E₁₅, E₁₆, E₁₇, E₁₈, E₁₉ and E₂₀. In one example, the hydrogen oxidizing bacterium may be genetically modified to express E₁ₐE_{1b}E_{1c}E_{1d} and at least one enzyme selected from E₃ to E₂₀ or all enzymes E₃ to E₂₀. In particular, the hydrogen oxidizing bacterium according to any aspect of the present invention may overexpress E₁ₐE_{1b}E_{1c}E_{1d} in combination with at least E₆.

Unless otherwise mentioned, all technical and scientific terms used herein have the same meaning as commonly understood by a skilled person. Any method known in the art may be used by the skilled person. Some examples of these methods are provided in the present description. The materials, methods, and examples are illustrative only and not intended to be limiting.

The accession numbers used throughout this description are derived from the NCBI database (National Centre for Biotechnology Information) maintained by the National Institute of Health, U.S.A. The accession numbers are as provided in the database on 30 Jun. 2014. Similarly, the EC numbers provided throughout this description are derived from the KEGG Ligand database, maintained by the Kyoto Encyclopedia of Genes and Genomics, sponsored in part by the University of Tokyo. The EC numbers are as provided in the database on 30 Jun. 2014.

Having generally described this invention, a further understanding can be obtained by reference to certain specific examples which are provided herein for purposes of illustration only, and are not intended to be limiting unless otherwise specified.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Construction of Plasmids for the Preparation of Fatty Acids with Ralstonia eutropha

1. The ribosome binding site of the R. eutropha groEL gene (SEQ ID NO: 1), the gene encoding the thioesterase from Cuphea hookeriana ChFATB2 (SEQ ID NO: 2) which has been 5′-truncated at the plastid targeting sequence, the terminator of the E. coli rrnB gene (SEQ ID NO: 5), wherein the coding region for ChFATB2 in the translation of R. eutropha is codon optimised (RBS RegroEL-ChFATB2-T; SEQ ID NO: 6).
2. The ribosome binding site of the R. eutropha groEL gene (SEQ ID NO: 1), the gene encoding the thioesterase of Cocos nucifera CnFATB3 (SEQ ID NO:3), the terminator of the E. coli rrnB gene (SEQ ID NO: 5), wherein the coding region for ChFATB3 in the translation of R. eutropha is codon optimised (RBS RegroEL-ChFATB3-T; SEQ ID NO: 7).
3. The ribosome binding site of the R. eutropha groEL gene (SEQ ID NO: 1), the gene encoding the thioesterase of Umbellularia californica UcFATB1 (SEQ ID NO: 4) the terminator of the E. coli rrnB gene (SEQ ID NO: 5), wherein the coding region for UcFATB1 in the translation of R. eutropha is codon optimised (RBS RegroEL-UCTE-T; SEQ ID NO: 8).

The expression cassettes RBS RegroEL-ChFATB2-T, RBS RegroEL-CnFATB3-T and RBS RegroEL-UcFATB1-T were then cloned via KpnI/HindIII in the broad host range expression vector pBBR1MCS-2 (SEQ ID NO: 9), so that expression of the genes was under the control of the E. coli lacZ promoter. The resulting expression plasmids were designated as PbBr-ChFATB2, PbBr-CnFATB3 and PbBr-UcFATB1 with respective sequences, SEQ ID NOs: 10, 11 and 12.

### Example 2

### Introducing Plasmids for the Production of Fatty Acids in Ralstonia eutropha

The plasmids from above were transfected into competent E. coli S17-1 cells, a strain where the conjugative transfer of plasmids from Ralstonia eutropha among other strains is possible. For this purpose, a Spotmating conjugation (as in FRIEDRICH et al, 1981) with the respective plasmids was carried out where E. coli S17-1 strain is the donor and R. eutropha H16 (reclassified as Alcaligenes eutrophus, DSMZ 428) and R. eutropha PHB-4 (reclassified as Alcaligenes eutrophus, DSMZ 541) the recipient. Transconjugants were obtained in all cases which carry the respective plasmids and the corresponding strains designated as follows:
R. eutropha H16 PbBr-ChFATB2, R. eutropha H16 PbBr-CnFATB3, R. eutropha H16 PbBr-UcFATB1, R. eutropha PHB-4-PbBr ChFATB2, R. eutropha PHB-4-PbBr CnFATB3, and R. eutropha PHB-4-PbBr UcFATB1.

### Example 3

### Quantification of Fatty Acids

Quantification of octanoic acid, 3-hydroxydecanoic acid, decanoic acid, lauric acid, 3-hydroxymyristic acid, myristic acid, palmitoleic acid, palmitic acid, oleic acid and stearic acid in the fermentation samples is performed by HPLC-ESI/MS based on an internal calibration for all analytes and using the internal standard D3 lauric acid (methyl-D3, 99%) of octanoic acid, 3-hydroxydecanoic acid, decanoic acid, lauric acid, 3 hydroxymyristic acid, myristic acid, palmitoleic acid, stearic acid, and D3 (D3-methyl, 98%) of palmitic acid, oleic acid, stearic acid.

The following devices are used:
HPLC system: Surveyor (Thermo Fisher Scientific, Waltham, Mass., USA), consisting of Surveyor MS Pump, Surveyor Autosampler Plus and Surveyor Surveyor PDA
mass spectrometer: TSQ Vantage HESI II—source (Thermo Fisher Scientific, Waltham, Mass., USA)
HPLC column: XBridge BEH C8, 100×2.1 mm, particle size: 2.5 microns, pore size 130 Å (Waters, Milford Mass., USA)

The samples are prepared by mixing 1200 μl of acetone and 300 μl of the sample for about 10 seconds and then centrifuged at about 13,000 rpm for 5 min. The clear supernatant is removed and analysed after appropriate dilution with acetone. For each 900 μl of the diluted sample 100 μl of ISTD solution is pipetted in.

HPLC separation is carried out using the above HPLC column. The injection volume is 2 μl, the column temperature 25° C., flow rate 0.3 mL/min. The mobile phase consists of eluent A (water+10 mM ammonium acetate adjusted to pH=9 neutralized with ammonia) and eluent B (acetonitrile/mobile phase A 95/5). The following gradient is used:

| Time [min] | Eluent A [%] | Eluent B [%] |
|---|---|---|
| 0 | 95 | 5 |
| 1 | 95 | 5 |
| 1.1 | 70 | 30 |
| 7 | 5 | 95 |
| 8 | 5 | 95 |

ESI-MS analysis is carried out in negative ionization with the following parameters from the ESI source:
Spray Voltage: 3000 V
Vaporizer Temperature: 380° C.
Sheath gas pressure: 40
Aux Gas Pressure: 15
Capillary Temperature: 380° C.

The detection and quantification of individual compounds is made by ‘single ion monitoring (SIM)’ with the following parameters:

| | Ion | Scan | Scan | Peak- |
|---|---|---|---|---|
| | [M − H]⁻ | width | Time | Weite |
| Analyte | [m/z] | [m/z] | [ms] | Q₃ |
| Octanoic acid | 143.13 | 0.002 | 100 | 0.7 |
| 3-hydroxydecanoic acid | 187.13 | 0.002 | 50 | 0.7 |
| decanoic acid | 171.13 | 0.002 | 100 | 0.7 |
| lauric acid | 199.16 | 0.002 | 50 | 0.7 |
| 3-hydroxymyristic acid | 243.18 | 0.002 | 50 | 0.7 |
| myristic acid | 227.19 | 0.002 | 50 | 0.7 |
| Palmitoleic acid | 253.18 | 0.002 | 50 | 0.7 |
| palmitic acid | 255.22 | 0.002 | 30 | 0.7 |
| oleic acid | 281.23 | 0.002 | 30 | 0.7 |
| stearic acid | 283.25 | 0.002 | 30 | 0.7 |
| D3-lauric acid | 202.16 | 0.002 | 50 | 0.7 |
| D3-stearic acid | 286.25 | 0.002 | 30 | 0.7 |

After cultivation of the following strains, the formation of fatty acids can be detected:
R. eutropha H16 pBBR-ChFATB2
R. eutropha H16 pBBR-CnFATB3
R. eutropha H16 pBBR-UcFATB1
R. eutropha PHB-4 pBBR-ChFATB2
R. eutropha PHB-4 pBBR-CnFATB3
R. eutropha PHB-4 pBBR-UcFATB1

### Example 4

### Usage of Genetic Modified Cells for Fatty Acids Production Via Usage of Knallgas

Genetic modified cells for fatty acids formation:
R. eutropha H16 pBBR-ChFATB2
R. eutropha H16 pBBR-CnFATB3
R. eutropha H16 pBBR-UcFATB1
R. eutropha PHB-4 pBBR-ChFATB2
R. eutropha PHB-4 pBBR-CnFATB3
R. eutropha PHB-4 pBBR-UcFATB1

All strains are cultured in 2×250 ml shaking flask with baffles in 25 ml medium according to Vollbrecht et al. 1978.

The medium contains (NH₄)2HPO₄2.0 g/l; KH₂PO₄ 2.1 g/l; MgSO₄×7 H₂O 0.2 g/l; FeCl₃×6H₂0 6 mg/l; CaCl₂×2H₂0 10 mg; trace element solution (Pfennig and Lippert, 1966) 0.1 ml. The trace element solution contains Titriplex III 10 g/l, FeSO₄×7H₂O 4 g/l, ZnSO₄×7H₂O, 0.2 g/l, MnCl₂×4H₂O 60 mg/l, H₃BO₃ 0.6 g/l, CoCl₂×6H₂O 0.4 g/l, CuCl₂×2H₂O 20 mg/l, NiCl₂×6H₂O 40 mg/l, Na₂Mo₄×2H₂O 60 mg/l. The medium of the preculture is supplemented with fructose 5 g/l, kanamycin 300 μg/ml and is inoculated from a cryoculture (1% (v/v)). The cultivation is performed at 30° C. and 150 rpm for 24 h.

Product formation is carried out in a 2 l stainless steel reactor Biostat B from Satorius under chemolithoautotrophic conditions with 1 l medium containing: (NH₄)2HPO₄ 2.0 g/l, KH₂PO₄ 2.1 g/l, MgSO₄×7H₂O 3 g/l, FeCl₃×6 H₂O 6 mg/l, CaCl₂×2 H₂O, 10 mg, biotin 1 mg/l, thiamin-HCl 1 mg/l, Ca-pantothenate 1 mg/l, nicotinic acid 20 mg/l, trace element solution 0.1 ml und polypropylenglycol (PPG 1000 diluted 1:5 with water adjusted at 30° C.).

Cultivation conditions are 30° C., 500-1500 rpm. The cultivation time is 76-150 h. The pH of 7 is adjusted with 1M NaOH. Gassing occurs with a gas mixture containing H₂ 90 vol %, CO₂ 6 vol %, O₂ 4 vol % with an overpressure of 0-2 bar and an aeration rate of 1.9 vvm.

The required volume of the preculture for inoculating the reactor (0.1% (v/v)) is harvested by centrifuging in 50 ml falcon tubes (10 min at 20° C. and 4500 rpm). In a washing step, which is repeated 3 times, the pellet is dissolved in 10 ml phosphate buffered saline and the suspension is centrifuged again (10 min at 20° C. and 4500 rpm). Finally the pellet is dissolved in 10 ml of medium ((NH₄)2HPO₄ 2.0 g/l, KH₂PO₄ 2.1 g/l, MgSO₄×7 H₂O 3 g/l, FeCl₃×6 H₂O 6 mg/l, CaCl₂×2 H₂O, 10 mg, biotin 1 mg/l, thiamin-HCl 1 mg/l, Ca-pantothenate 1 mg/l, nicotinic acid 20 mg/l, trace element solution 0.1 ml und polypropylenglycol (PPG 1000 diluted 1:5 with water adjusted at 30° C.).

After cultivation the concentrations of fatty acids are determined as described.

European patent application EP14193474 filed Nov. 17, 2014, is incorporated herein by reference.

Numerous modifications and variations on the present invention are possible in light of the above teachings. It is therefore to be understood that within the scope of the appended claims, the invention may be practiced otherwise than as specifically described herein.

1. A method for producing at least one fatty acid and/or derivative thereof from a gas comprising H₂, CO₂, and O₂, the method comprising:
(a) providing a genetically modified hydrogen oxidizing bacterium in an aqueous medium; and
(b) contacting the aqueous medium with said gas comprising H₂, CO₂, and O₂, in a weight ratio of 20 to 70 (H₂): 10 to 45 (CO₂): 5 to 35 (O₂);
wherein the fatty acid comprises at least 5 carbon atoms; and
wherein the hydrogen oxidizing bacterium is genetically modified relative to the wild type bacterium to increase the expression of enzyme E₁ that catalyzes the conversion of acetyl CoA to acyl ACP via malonyl CoA and to increase the expression of enzyme E₂ that catalyzes the conversion of Acyl ACP to the fatty acid.

2. The method according to claim 1, wherein E₁ is selected from the group consisting of E₁ₐ accA (EC 6.4.1.2), E_{1b} accB (EC 6.4.1.2), E_{1c} accC (EC 6.4.1.2) and E_{1d} accD (EC 6.4.1.2) and combinations thereof, and/or E₂ is at least one thioesterase (EC 3.1.2.14 and EC 3.1.2.22).

3. The method according to claim 1, wherein the hydrogen oxidizing bacterium is genetically modified relative to the wild type bacterium to increase the expression of enzyme E₁ₐ, E_{1b}, E_{1c} and E_{1d}.

4. The method according to claim 3, wherein
(a) E₁ₐ has sequence identity of at least 50% to a polypeptide with accession number AAC73296 or EG11647, and combinations thereof,
(b) E_{1b} has sequence identity of at least 50% to a polypeptide with accession number EG10275 and combinations thereof,
(c) E_{1c} has sequence identity of at least 50% to a polypeptide with accession number EG10276 and combinations thereof, and
(d) E_{1d} has sequence identity of at least 50% to a polypeptide of with accession number EG10217 and combinations thereof,
or to a functional fragment of any of the polypeptides for catalyzing the conversion of conversion of acetyl CoA to acyl ACP via malonyl CoA.

5. The method according to any one of claim 1, wherein E₂ is an acyl-ACP thioesterase.

6. The method according to claim 5, wherein E₂ has sequence identity of at least 50% to a polypeptide with accession number selected from the group consisting of AAC72881.1, ABB71579.1, CAC19934.1, AAC49180.1, AAC49783.1, AAC49179.1, CAB60830.1, ABB71581.1, AAC49269.1, CAC19933.1, CAA54060.1, AAC72882.1, Q39513.1, AAC49784.1, AAC72883.1, Q41635.1, and AAC49001.1 and combinations thereof.

7. The method according to claim 1, wherein the hydrogen oxidizing bacterium is further genetically modified relative to the wild type bacterium to increase the expression of at least one enzyme selected from the group consisting of E₃ aceE (EC 1.2.4.1, 2.3.1.61, 2.3.1.12), E₄ aceF (EC 1.2.4.1, 2.3.4.16, 2.3.1.12), E₅ acpP (AAC74178), E₆ fadD (EC 2.3.1.86), E₇ cerl (EC 4.1.99.5), E₈ fabA (EC4.2.1.60), E₉ fabB (EC 2.3.1.41), E₁₀ fabD (EC 2.3.1.39), E₁₁ fabG (EC 1.1.1.100), E₁₂ fabH (EC 2.3.1.180), E₁₃ fabl (EC 1.3.1.9), E₁₄ fabZ (EC 4.2.1.-), E₁₅ lipase (EC 3.1.1.3), E₁₆ malonyl-CoA decarboxylase (EC 4.1.1.9, 4.1.1.41), E₁₇ panD (EC 4.1.1.11), E₁₈ panK (EC 2.7.1.33), E₁₉ pdh (EC 1.2.4.1), E₂₀ udhA (EC 1.6.1.1) and combinations thereof.

8. The method according to claim 1, wherein the hydrogen oxidizing bacteria is selected from the group consisting of Achromobacter, Acidithiobacillus, Acidovorax, Alcaligenes, Anabena, Aquifex, Arthrobacter, Azospirillum, Bacillus, Bradyrhizobium, Cupriavidus, Derxia, Helicobacter, Herbaspirillum, Hydrogenobacter, Hydrogenobaculum, Hydrogenophaga, Hydrogenophilus, Hydrogenothermus, Hydrogenovibrio, Ideonella sp. 01, Kyrpidia, Metallosphaera, Methanobrevibacter, Myobacterium, Nocardia, Oligotropha, Paracoccus, Pelomonas, Polaromonas, Pseudomonas, Pseudonocardia, Rhizobium, Rhodococcus, Rhodopseudomonas, Rhodospirillum, Streptomyces, Thiocapsa, Treponema, Variovorax, Xanthobacter and Wautersia.

9. The method according to claim 1, wherein the hydrogen oxidizing bacterium is further genetically modified to increase the expression relative to the wild type bacterium of enzyme E₂₁ a wax ester synthase (EC 2.3.1.75).

10. The method according to claim 1, wherein the hydrogen oxidizing bacterium is further genetically modified to increase the expression relative to the wild type bacterium of enzyme E₂₆ a fatty acid methyl transferase (EC 2.1.1.15).

11. The method according to claim 1, wherein the bacterium comprises an exogenous nucleic sequence encoding ACP, Sfa, or combinations thereof.

12. The method according to claim 2, wherein E₂ is at least one thioesterase (EC 3.1.2.14 and EC 3.1.2.22).

13. The method according to claim 2, wherein E₂ is at least one thioesterase (EC 3.1.2.14).

14. The method according to claim 2, wherein E₂ is at least one thioesterase (EC 3.1.2.22).


SEQUENCE LISTING (12 sequences)

SEQ ID NO: 1 - DNA, 17 bp, Ralstonia eutropha
tttcaggagattcaaga

SEQ ID NO: 2 - protein, 305 aa, Cuphea hookeriana
MHDRKSKRPDMLVDSFGLESTVQDGLVFRQSFSIRSYEIGTDRTASIETLMNHLQETSLNHCKSTGILLDGFGRTLEMCKRDLIWVVIKMQIKVNRYPAWGDTVEINTRFSRLGKIGMGRDWLISDCNTGEILVRATSAYAMMNQKTRRLSKLPYEVHQEIVPLFVDSPVIEDSDLKVHKFKVKTGDSIQKGLTPGWNDLDVNQHVSNVKYIGWILESMPTEVLETQELCSLALEYRRECGRDSVLESVTAMDPSKVGVRSQYQHLLRLEDGTAIVNGATEWRPKNAGANGAISTGKTSNGNSVS

SEQ ID NO: 3 - protein, 327 aa, Cocos nucifera
MPDWSMLLAAIRTIFSAAEKQWTLLDSKKRGADAVADASGVGKMVKNGLVYRQNFSIRSYEIGVDKRASVEALMNHFQETSLNHCKCIGLMHGGFGCTPEMTRRNLIWVVAKMLVHVERYPWWGDVVQINTWISSSGKNGMGRDWHVHDCQTGLPIMRGTSVWVMMDKHTRRLSKLPEEVRAEITPFFSERDAVLDDNGRKLPKFDDDSAAHVRRGLTPRWHDFDVNQHVNNVKYVGWILESVPVWMLDGYEVATMSLEYRRECRMDSVVQSLTAVSSDHADGSPIVCQHLLRLEDGTEIVRGQTEWRPKQQARDLGNMGLHPTESK

SEQ ID NO: 4 - protein, 301 aa, Umbellularia californica
MTLEWKPKPKLPQLLDDHFGLHGLVFRRTFAIRSYEVGPDRSTSILAVMNHMQEATLNHAKSVGILGDGFGTTLEMSKRDLMWVVRRTHVAVERYPTWGDTVEVECWIGASGNNGMRRDFLVRDCKTGEILTRCTSLSVLMNTRTRRLSTIPDEVRGEIGPAFIDNVAVKDDEIKKLQKLNDSTADYIQGGLTPRWNDLDVNQHVNNLKYVAWVFETVPDSIFESHHISSFTLEYRRECTRDSVLRSLTTVSGGSSEAGLVCDHLLQLEGGSEVLRARTEWRPKLTDSFRGISVIPAEPRV

SEQ ID NO: 5 - DNA, 57 bp, Escherichia coli
tagtcaaaagcctccggtcggaggcttttgactttctgcttactgaatttgcggccg

SEQ ID NO: 6 - DNA, 1004 bp, Artificial Sequence
  codon optimised gene
ggtacctttcaggagattcaagaatgcatgatcggaaatccaaacgccccgatatgctcgtggactcgttcggcctggaaagcaccgtccaggatgggctggtgtttcggcaatcgttttcgatccggagctatgaaattggcaccgaccggacggcgagcattgaaacgctgatgaaccatctgcaggaaacctccctgaaccactgcaaatccaccggcattctcctcgatgggtttgggcgcacgctcgaaatgtgtaagcgcgatctgatctgggtggtgattaagatgcagattaaagtgaaccggtatcccgcgtggggcgacaccgtggagatcaatacccggttttcgcgcctcgggaagattgggatggggcgggactggctgattagcgactgcaacacgggcgagattctcgtccgggccacctcggcctatgccatgatgaatcagaaaacccgccggctgagcaaactgccctacgaagtgcaccaggaaatcgtcccgctcttcgtcgactccccggtgatcgaagattcggacctgaaggtgcataagttcaaggtgaagacgggggatagcattcagaagggcctgacgccgggctggaacgatctcgacgtgaaccagcatgtgtccaacgtcaaatacatcggctggattctggaatcgatgccgacggaagtcctggagacccaagaactgtgctccctcgccctcgagtatcgccgggagtgtggccgcgactcggtcctcgaatcggtgaccgccatggacccgtcgaaggtcggggtgcggtcgcaatatcaacacctgctgcgcctggaggacgggaccgcgattgtgaacggcgccaccgagtggcggccgaaaaatgcgggggccaacggggccattagcacgggcaagacgagcaacggcaactccgtgtcgtaatagtcaaaagcctccggtcggaggcttttgactttctgcttactgaatttgcggccgaagctt

SEQ ID NO: 7 - DNA, 1070 bp, Artificial Sequence
  codon optimised gene
ggtacctttcaggagattcaagaatgccggactggtcgatgctgctggcggccatccgcaccattttcagcgccgcggagaagcagtggaccctgctggactcgaagaagcggggggcggacgcggtggccgacgcctccggggtgggcaagatggtgaagaatggcctggtgtaccgccagaacttctcgatccgctcgtatgagatcggggtggacaagcgcgcctcggtcgaggccctgatgaaccacttccaggagacctcgctcaaccactgcaagtgcatcggcctgatgcatggcggcttcggctgcacgccggagatgacgcggcgcaacctgatctgggtcgtggcgaagatgctggtgcacgtggagcgctatccgtggtggggcgacgtggtgcagattaacacctggatctcgtcgtccgggaagaacggcatgggccgggattggcatgtgcacgattgccaaaccggcctgccgatcatgcgcgggacgtcggtgtgggtgatgatggacaagcacacccggcggctctcgaagctgccggaggaggtgcgcgcggaaatcaccccgtttttctcggagcgcgatgcggtgctcgatgacaacggccgcaagctgccgaagttcgacgatgactccgccgcgcatgtgcggcgcgggctgaccccgcgctggcatgattttgacgtgaatcagcatgtgaacaacgtgaagtacgtgggctggattctggagtccgtgcccgtgtggatgctcgacgggtacgaggtggccacgatgtcgctggagtatcgccgcgagtgccgcatggactccgtggtgcaatcgctgaccgccgtgtcgtcggaccacgccgacggctcgccgatcgtgtgccagcacctgctgcgcctggaggacggcaccgaaattgtgcggggccagacggagtggcgcccgaaacagcaggcccgcgacctgggcaacatggggctgcacccgacggagtcgaagtaatagtcaaaagcctccggtcggaggcttttgactttctgcttactgaatttgcggccgaagctt

SEQ ID NO: 8 - DNA, 992 bp, Artificial Sequence
  codon optimised gene
ggtacctttcaggagattcaagaatgaccctcgaatggaagccgaaaccgaagctgccgcagctcctggacgatcattttggcctgcacgggctggtgttccgccgcacgttcgccatccgctcgtatgaagtcggccccgaccggagcacgtccatcctggcggtgatgaatcacatgcaagaggccaccctgaaccatgccaaatccgtgggcatcctcggcgacggctttggcaccacgctggagatgtcgaaacgcgacctgatgtgggtggtccgccggacccatgtggccgtggagcgctacccgacctggggcgacaccgtggaagtggagtgctggatcggcgcctcgggcaacaacggcatgcgccgggatttcctggtgcgcgactgcaagaccggcgagatcctcacgcgctgcacctcgctgtcggtgctcatgaatacccgcacgcgccgcctgtcgaccattccggacgaagtccgcggggagattggcccggcgttcattgataatgtcgcggtgaaggacgacgagatcaagaaactccagaagctgaacgactcgaccgccgactacatccaagggggcctgacgccccggtggaacgatctcgatgtgaaccagcacgtgaacaacctcaaatatgtggcgtgggtgtttgagaccgtgccggactcgatcttcgaatcgcatcacatctcgagctttaccctggagtatcgccgcgagtgcacccgggacagcgtcctgcgctcgctcaccaccgtcagcggcgggtcgtcggaagcggggctcgtgtgcgaccacctcctgcagctggagggggggtccgaagtgctgcgcgcccggaccgagtggcggcccaaactgaccgattccttccgcggcatttcggtcatccccgcggagccgcgggtgtaatagtcaaaagcctccggtcggaggcttttgactttctgcttactgaatttgcggccgaagctt

SEQ ID NO: 9 - DNA, 5144 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 10 - DNA, 6106 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttcggccgcaaattcagtaagcagaaagtcaaaagcctccgaccggaggcttttgactattacgacacggagttgccgttgctcgtcttgcccgtgctaatggccccgttggcccccgcatttttcggccgccactcggtggcgccgttcacaatcgcggtcccgtcctccaggcgcagcaggtgttgatattgcgaccgcaccccgaccttcgacgggtccatggcggtcaccgattcgaggaccgagtcgcggccacactcccggcgatactcgagggcgagggagcacagttcttgggtctccaggacttccgtcggcatcgattccagaatccagccgatgtatttgacgttggacacatgctggttcacgtcgagatcgttccagcccggcgtcaggcccttctgaatgctatcccccgtcttcaccttgaacttatgcaccttcaggtccgaatcttcgatcaccggggagtcgacgaagagcgggacgatttcctggtgcacttcgtagggcagtttgctcagccggcgggttttctgattcatcatggcataggccgaggtggcccggacgagaatctcgcccgtgttgcagtcgctaatcagccagtcccgccccatcccaatcttcccgaggcgcgaaaaccgggtattgatctccacggtgtcgccccacgcgggataccggttcactttaatctgcatcttaatcaccacccagatcagatcgcgcttacacatttcgagcgtgcgcccaaacccatcgaggagaatgccggtggatttgcagtggttcagggaggtttcctgcagatggttcatcagcgtttcaatgctcgccgtccggtcggtgccaatttcatagctccggatcgaaaacgattgccgaaacaccagcccatcctggacggtgctttccaggccgaacgagtccacgagcatatcggggcgtttggatttccgatcatgcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 11 - DNA, 6172 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttcggccgcaaattcagtaagcagaaagtcaaaagcctccgaccggaggcttttgactattacttcgactccgtcgggtgcagccccatgttgcccaggtcgcgggcctgctgtttcgggcgccactccgtctggccccgcacaatttcggtgccgtcctccaggcgcagcaggtgctggcacacgatcggcgagccgtcggcgtggtccgacgacacggcggtcagcgattgcaccacggagtccatgcggcactcgcggcgatactccagcgacatcgtggccacctcgtacccgtcgagcatccacacgggcacggactccagaatccagcccacgtacttcacgttgttcacatgctgattcacgtcaaaatcatgccagcgcggggtcagcccgcgccgcacatgcgcggcggagtcatcgtcgaacttcggcagcttgcggccgttgtcatcgagcaccgcatcgcgctccgagaaaaacggggtgatttccgcgcgcacctcctccggcagcttcgagagccgccgggtgtgcttgtccatcatcacccacaccgacgtcccgcgcatgatcggcaggccggtttggcaatcgtgcacatgccaatcccggcccatgccgttcttcccggacgacgagatccaggtgttaatctgcaccacgtcgccccaccacggatagcgctccacgtgcaccagcatcttcgccacgacccagatcaggttgcgccgcgtcatctccggcgtgcagccgaagccgccatgcatcaggccgatgcacttgcagtggttgagcgaggtctcctggaagtggttcatcagggcctcgaccgaggcgcgcttgtccaccccgatctcatacgagcggatcgagaagttctggcggtacaccaggccattcttcaccatcttgcccaccccggaggcgtcggccaccgcgtccgccccccgcttcttcgagtccagcagggtccactgcttctccgcggcgctgaaaatggtgcggatggccgccagcagcatcgaccagtccggcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 12 - DNA, 6094 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttcggccgcaaattcagtaagcagaaagtcaaaagcctccgaccggaggcttttgactattacacccgcggctccgcggggatgaccgaaatgccgcggaaggaatcggtcagtttgggccgccactcggtccgggcgcgcagcacttcggaccccccctccagctgcaggaggtggtcgcacacgagccccgcttccgacgacccgccgctgacggtggtgagcgagcgcaggacgctgtcccgggtgcactcgcggcgatactccagggtaaagctcgagatgtgatgcgattcgaagatcgagtccggcacggtctcaaacacccacgccacatatttgaggttgttcacgtgctggttcacatcgagatcgttccaccggggcgtcaggcccccttggatgtagtcggcggtcgagtcgttcagcttctggagtttcttgatctcgtcgtccttcaccgcgacattatcaatgaacgccgggccaatctccccgcggacttcgtccggaatggtcgacaggcggcgcgtgcgggtattcatgagcaccgacagcgaggtgcagcgcgtgaggatctcgccggtcttgcagtcgcgcaccaggaaatcccggcgcatgccgttgttgcccgaggcgccgatccagcactccacttccacggtgtcgccccaggtcgggtagcgctccacggccacatgggtccggcggaccacccacatcaggtcgcgtttcgacatctccagcgtggtgccaaagccgtcgccgaggatgcccacggatttggcatggttcagggtggcctcttgcatgtgattcatcaccgccaggatggacgtgctccggtcggggccgacttcatacgagcggatggcgaacgtgcggcggaacaccagcccgtgcaggccaaaatgatcgtccaggagctgcggcagcttcggtttcggcttccattcgagggtcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt